(19) United States
(12) Patent Application Publication
(10) Pub. No.: US 2025/0143827 A1
(43) Pub. Date: May 8, 2025

(54) Resilient Drape Assemblies With Guides For Use With Surgical Robots

(71) Applicant: Mobius Imaging, LLC, Shirley, MA (US)

(72) Inventors: Edward Daley, Maynard, MA (US); Russell Stanton, Lunenberg, MA (US); Gordon Row, Groton, MA (US)

(73) Assignee: Mobius Imaging, LLC, Shirley, MA (US)

(21) Appl. No.: 18/836,832

(22) PCT Filed: Feb. 8, 2023
(86) PCT No.: PCT/US2023/012570

§ 371 Date: Aug. 8, 2024

Related U.S. Application Data
(60) Provisional application No. 63/307,823, filed on Feb. 8, 2022.

(51) Int. Cl. A61B 46/10; A61B 34/20; A61B 46/00

(52) U.S. Cl. CPC A61B 46/10; A61B 34/20; A61B 46/40; A61B 2034/2055

(57) ABSTRACT

The present teachings generally provide for a robotic surgical system also including a robotic arm extending between a distal end and a proximal end; a keeper coupled to the robotic arm, with the keeper having a keeper interface. The system further including a drape including: a first portion defining an opening shaped to receive the distal end of the robotic arm there through, a second portion disposed in fluid communication with the first portion and defining a cavity shaped to accommodate at least a portion of the distal end of the robotic arm therein, and a guide disposed, between the first portion and the second portion and having a guide interface configured to releasably attach to the keeper interface of the keeper to secure the drape to the robotic arm.

### CROSS-REFERENCE TO RELATED APPLICATION

The subject patent application claims priority to and all the benefits of U.S. Provisional Patent Application No. 63/307,923, filed on Feb. 8, 2022, the disclosure of which is hereby incorporated by reference in its entirety.

### BACKGROUND

Sterile barrier assemblies such as surgical drapes are known for establishing barriers between surgical components during surgery. For instance, a surgical drape may be used to provide a barrier between a robotic arm and a tool attached to the robotic arm. In surgery, the robotic arm is treated as being nonsterile, while the tool is sterile. The surgical drape creates a barrier between the robotic arm and the tool to prevent contamination of a sterile field in which the tool is operating. Depending on the specific configuration of the robotic arm, it can sometimes be difficult to install the surgical drape while simultaneously maintaining desired arrangements of different sterile barriers, zones, fields, and the like. For example, imprecise handling of the surgical drape may inadvertently result in an external surface of the surgical drape coming into contact with a non-sterile surface (e.g., the outer surface of the robotic arm). In general terms, if the sterile barrier defined by the surgical drape is compromised, standard operating room sterility protocol may dictate that the surgical drape requires replacement, which takes up valuable time.

Certain conventional types of surgical drapes may be placed between the robotic arm and a sterile end effector which supports the tool, or may be placed over the robotic arm and the end effector. These types of conventional surgical drapes may include one or more perforations or other openings through which different connections can be made between the robotic arm, such as mechanical connections and/or electrical connections. Perforations may also be utilized adjacent to light emitting diodes (LEDs) employed along the robotic arm for use with navigation systems which employ optical sensing technology to monitor, track, or otherwise facilitate control of the robotic arm. In addition to and/or in place of perforations, certain types of drape assemblies necessitate that external fasteners, such as locators, locks, diffusers, plates, clips, and the like (and/or “windows” of transparent material), be located and placed over the surgical drape adjacent to the LEDs in order to ensure that light emitted by the LEDs can be properly observed by the navigation system. Here, for example, if the surgical drape is misaligned or otherwise not secured properly, the LEDs may create glare or otherwise not be properly visible and navigation errors can occur. It will be appreciated that when multiple LEDs are utilized along different portions of the robotic arm and/or end effector, it can sometimes be difficult to properly apply or otherwise install the surgical drape while aligning the external fasteners relative to their corresponding LEDs.

Beyond being cumbersome to install at the onset of a surgical procedure, the use of these types of external fasteners can be problematic if they become loose or otherwise need to be replaced intraoperatively. For example, if an external fastener which serves as a diffuser for an LED becomes misaligned or presents navigation errors during a surgical procedure, great care must be taken to remove and replace it without compromising the sterile barrier defined by the drape itself, as perforations, apertures, and the like are generally formed adjacent to the LEDs or may be formed during the process of initially installing the external fastener. Other surgical drapes are not intentionally perforated, but instead are compressed between the robotic arm and the external fasteners to facilitate proper diffusion of the LEDs. When compressed, if the surgical drape is formed of thin plastic, unintended rips or tears may occur.

Accordingly, there remains a need in the art for addressing one or more of the deficiencies described above.

### SUMMARY

The present teachings generally provide in one example for a robotic surgical system also including a robotic arm extending between a distal end and a proximal end; a keeper coupled to the robotic arm, with the keeper having a keeper interface. The system further including a drape including: a first portion defining an opening shaped to receive the distal end of the robotic arm therethrough, a second portion disposed in fluid communication with the first portion and defining a cavity shaped to accommodate at least a portion of the distal end of the robotic arm therein, and a guide disposed between the first portion and the second portion and having a guide interface configured to releasably attach to the keeper interface of the keeper to secure the drape to the robotic arm.

The teachings further provide for another example directed to a drape assembly configured for a robotic arm of a robotic surgical system. The drape assembly includes a keeper for securing the drape assembly to the robotic arm, the keeper having a keeper interface. The assembly also includes a drape including: a first portion defining an opening shaped to receive a distal end of the robotic arm therethrough, a second portion disposed in fluid communication with the first portion and defining a cavity shaped to accommodate at least a portion of the distal end of the robotic arm therein, and a guide disposed between the first portion and the second portion and having a guide interface configured to releasably attach to the keeper interface of the keeper to secure the drape to the robotic arm.

The present teachings also provide for a further example directed towards a robotic surgical system including a robotic arm having a plurality of links between a distal end and a proximal end. The robotic arm having an end effector operatively attached to the distal end of the robotic arm, the end effector including a plurality of markers protruding from a surface of the end effector. The system also includes a drape including: a first portion defining an opening shaped to receive the distal end of the robotic arm therethrough, and a second portion disposed in fluid communication with the first portion and defining a cavity shaped to accommodate at least a portion of the distal end of the robotic arm therein, the second portion of the drape being formed from an elastic polyisoprene material shaped to cover the end effector and contact each of the plurality of markers.

The present teachings generally provide for another example providing for a method of using a robotic surgical system. The method also includes positioning an end effector of a robotic arm within a field of view of a localizer of a navigation system, the end effector having a plurality of markers. The method also includes covering the end effector with a drape formed from an elastic polyisoprene material in contact with each of the plurality of markers. The method also includes detecting, with the localizer of the navigation system, light emitted by the plurality of markers through the drape.

Each of the examples may include one or more of the features described below separately or in combination. The keeper interface of the keeper may include a seat; and the guide may include a retainer operatively attached to the guide and shaped to releasably engage the seat of the keeper. The guide may be at least partially formed from a resilient material. The keeper interface may include a taper having a ramped profile shaped to resiliently enlarge the guide as the guide is passed over the keeper. The guide may define an aperture having an inner perimeter larger than an outer perimeter of the keeper. The guide may define an airflow channel arranged between the outer perimeter of the keeper and the inner perimeter of the aperture of the guide. The second portion of the drape may be formed from an elastic polyisoprene material shaped to cover the end effector and contact each of the plurality of markers. The robotic surgical system may further include a navigation system configured to optically track the plurality of markers on the end effector through the second portion of the drape. The first portion of the drape may include: a distal section coupled to the guide, and a proximal section defining the opening. The first portion of the drape and the second portion of the drape may be formed from different materials. The second portion of the drape may be formed from an elastic polyisoprene material. The first portion of the drape and the second portion of the drape are formed from the same material. The keeper may include two keeper interfaces, where the guide may include two guide interfaces to respectively releasably attach to the two keeper interfaces. The keeper may be integrated into the robotic arm adjacent to the distal end. The keeper may be removably connected to the robotic arm adjacent to the distal end. The keeper interface of the keeper may include a seat and the guide may include a retainer operatively attached to the guide and shaped to releasably engage the seat of the keeper. The guide may include a first guide interface and a second guide interface, the first guide interface being spaced from the second guide interface, and the keeper may includes a first keeper interface and a second keeper interface, the first keeper interface being spaced from the second keeper interface. The first guide interface and the second guide interface may each be shaped to releasable engage one of the first keeper interface and the second keeper interface. Each of the plurality of markers may include a lens, with the second portion of the drape contacting and covering the lens of each of the plurality of markers. Each of the plurality of markers may include a light emitting diode.

### BRIEF DESCRIPTION OF THE DRAWINGS

FIG. 1 is a perspective view of one examples of a robotic surgical system, including a navigation system including a localizer to track states of trackers within a field of view, a patient tracker adapted for attachment relative to a target site, and a robotic arm configured to maintain alignment of a tool relative to the target site.

FIG. 2 is a perspective view of the robotic arm of FIG. 1, with the robotic arm extending between a base end and a mount end arranged for movement relative to the base end, with an end effector attached to the mount end of the robotic arm.

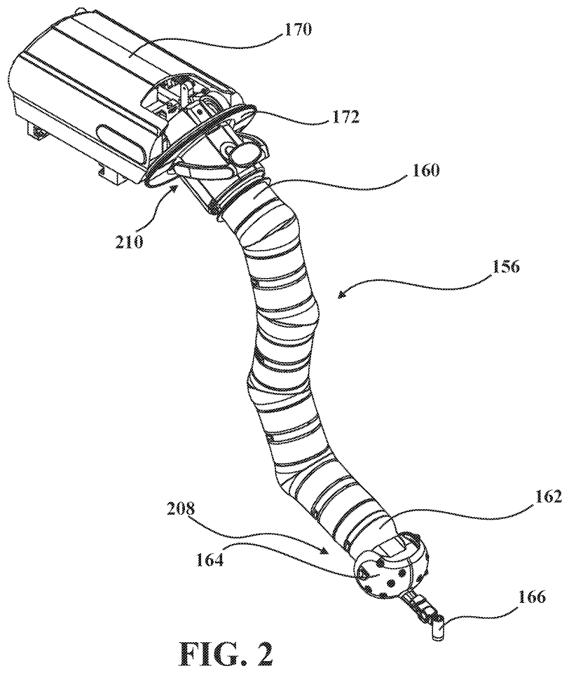

FIG. 3 is a perspective view depicting portions of a surgical system according to aspects of the present disclosure, shown with a drape assembly spaced from a robotic arm, the drape assembly having a guide coupled to a resilient drape and to a tube drape, and the robotic arm supporting an end effector and having a keeper.

FIG. 4A is another perspective view of the portions of the surgical system of FIG. 1, shown with the drape assembly depicted in phantom outline.

FIG. 4B is another perspective view of the portions of the surgical system of FIG. 4A, shown with the drape assembly depicted in phantom outline and secured to the robotic arm via engagement between the guide of the drape assembly and the keeper of the robotic arm.

FIG. 5 is another perspective view of the portions of the surgical system of FIG. 4B, shown with the drape assembly secured to the robotic arm.

FIG. 6 is a partial perspective view of the drape assembly of FIGS. 3-5 shown with the guide and the resilient drape of the drape assembly arranged adjacent to the keeper of the robotic arm.

FIG. 7A is another partial perspective view of the drape assembly and the keeper of FIG. 6, shown with the resilient drape and the tube drape depicted in phantom outline and with the guide spaced from the keeper.

FIG. 7B is another partial perspective view of the drape assembly and the keeper of FIG. 7A, shown with the guide engaging the keeper to secure the drape assembly to the robotic arm.

FIG. 7C is another partial perspective view of the drape assembly and the keeper of FIG. 7B, shown with the guide engaging the keeper and with the tube drape extended.

FIG. 8 is a front side plan view of the guide and the keeper of the surgical system.

FIG. 9 is a partial section view taken along line 9-9 in FIG. 8.

FIG. 10A is a perspective view depicting the guide of the drape assembly spaced proximally from the keeper of the robotic arm.

FIG. 10B is another perspective view of the guide and the keeper of FIG. 10A, shown with the guide positioned distally of the keeper in exaggerated fashion for illustrative purposes.

FIG. 10C is another perspective view of the guide and the keeper of FIGS. 10A-10B, shown with the guide engaging the keeper to secure the drape assembly to the robotic arm.

FIG. 11A is a partial perspective view of the end effector of the robotic arm of FIGS. 3-5, shown having light emitting diodes (LED) markers for tracking the robotic arm with a navigation system, and depicted with the resilient drape of the drape assembly positioned adjacent to the end effector.

FIG. 11B is another partial perspective view of the end effector of the robotic arm and the drape assembly of FIG. 11A, shown with the resilient drape of the drape assembly extended over the LED markers of the end effector.

FIG. 12 is a partial screenshot of a graphical user interface of a navigation system depicting visibility of the LED markers of the end effector through the resilient drape arranged as depicted in FIG. 11B.

### DETAILED DESCRIPTION

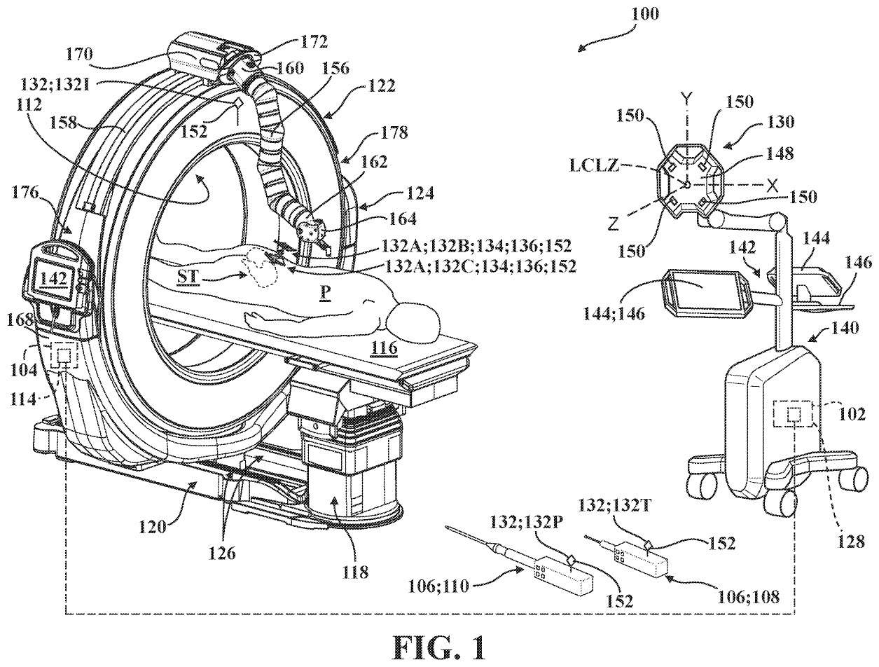

Referring now to the drawings, wherein like numerals indicate like or corresponding parts throughout the several views, a surgical system 100 is shown in FIG. 1 for treating a patient P. To this end, the illustrated surgical system 100 generally includes a navigation system 102, an imaging system 104, and one or more types of tools 106. As will be appreciated from the subsequent description below, the surgical system 100 is configured to, among other things, allow the surgeon to visualize, approach, and treat or otherwise manipulate anatomy of a patient P at a target site ST with a high level of control. To this end, imaging data of the target site ST may be acquired via the imaging system 104, and can be used to assist the surgeon in visualizing the patient's P anatomy at or otherwise adjacent to the target site ST. Here, the imaging data may also be utilized by the navigation system 102 to, among other things, facilitate navigation of tools 106 relative to the target site ST. Each of the components of the surgical system 100 introduced above will be described in greater detail below.

In FIG. 1, an operating room is shown with a patient P undergoing an exemplary surgical procedure performed using the surgical system 100. In this illustrative example, a minimally-invasive spinal surgical procedure, such as a posterior interbody spinal fusion, is being performed. It will be appreciated that this example is illustrative, and that other types of surgical procedures are contemplated. During the surgical procedure, one or more hand-held tools 106, such as a rotary tool 108 and/or a pointer tool 110, may be used by the surgeon. The tool 106 is for engaging the target site ST. As noted above and as is described in greater detail below, the navigation system 102 may be configured to track states of one or more of the tools 106 relative to the target site ST. In this exemplary surgical procedure, the rotary tool 108 may be employed as a cutting or drilling tool to remove tissue, form pilot holes (e.g., in the ilium, in vertebrae, and the like), or otherwise approach the target site ST. The rotary tool 108 may also be used to drive or otherwise install implantable components (e.g., pedicle screws, anchors, and the like).

For illustrative purposes, generically-depicted tools 106 configured for hand-held use are shown in FIG. 1. However, as will be appreciated from the subsequent description below, aspects of the surgical system 100 may be used with any suitable type of tool 106 without departing from the scope of the present disclosure. Furthermore, in addition to hand-held tools 106 of various types and configurations, aspects of the surgical system 100 may also be employed in connection with robotically-controlled tools 106 (not shown). Certain types of robotically-controlled tools 106 are disclosed in U.S. Pat. No. 9,119,655, entitled “Surgical Robotic arm Capable of Controlling a Surgical Instrument in Multiple Modes;” U.S. Pat. No. 10,456,207, entitled “Systems and Tools for use with Surgical Robotic Manipulators;” U.S. Pat. No. 11,160,620, entitled “End Effectors And Methods For Driving Tools Guided By Surgical Robotic Systems;” U.S. Pat. No. 10,959,783, entitled “Integrated Medical Imaging and Surgical Robotic System;” and U.S. Patent Application Publication No. 2020/0078097, entitled “Methods and Systems for Robot-Assisted Surgery,” the disclosures of each of which are hereby incorporated by reference in their entirety.

As noted above, the imaging system 104 may be used to obtain imaging data ID of the patient, which may be a human or animal patient. In the representative version illustrated in FIG. 1, the imaging system 104 is realized as an x-ray computed tomography (CT) imaging device. Here, the patient P may be positioned within a central bore 112 of the imaging system 104 and an x-ray source and detector may be rotated around the central bore 112 to obtain raw x-ray imaging data of the patient P. The imaging data may be processed using an imaging system controller 114, or another suitable controller, in order to construct three-dimensional imaging data, two-dimensional imaging data, and the like, which may be transmitted to or otherwise utilized by the navigation system 102 or other components of the surgical system 100.

In some versions, imaging data may be obtained preoperatively (e.g., prior to performing a surgical procedure) or intraoperatively (e.g., during a surgical procedure) by positioning the patient P within the central bore 112 of the imaging system 104. In order to obtain imaging data, a portion of the imaging system 104 may be moved relative to a patient support 116 (e.g., a surgical table) on which the patient P is disposed while the patient P remains stationary. Here, the patient support 116 is secured to the imaging system 104, such as via a column 118 which is mounted to a base 120 of the imaging system 104. A portion of the imaging system 104 (e.g., an O-shaped imaging gantry 122) which includes at least one imaging component may be supported by an articulable support 124 that can translate along the length of the base 120 on rails 126 to perform an imaging scan of the patient P, and may translate away from the patient P to an out-of-the-way position for performing a surgical procedure on the patient P.

An exemplary imaging system 104 that may be used in various versions is the AIRO® intra-operative CT system manufactured by Mobius Imaging, LLC. Examples of x-ray CT imaging devices that may be used according to various versions of the present disclosure are described in U.S. Pat. No. 10,151,810, entitled “Pivoting Multi-directional X-ray Imaging System with a Pair of Diametrically Opposite Vertical Support Columns Tandemly Movable Along a Stationary Base Support;” U.S. Pat. No. 9,962,132, entitled “Multi-directional X-ray Imaging System with Single Support Column;” U.S. Pat. No. 9,801,592, entitled “Caster System for Mobile Apparatus;” U.S. Pat. No. 9,111,379, entitled “Method and System for X-ray CT Imaging;” U.S. Pat. No. 8,118,488, entitled “Mobile Medical Imaging System and Methods;” and U.S. Patent Application Publication No. 2014/0275953, entitled “Mobile X-ray Imaging System,” the disclosures of each of which are hereby incorporated by reference in their entirety.

While the illustrated imaging system 104 is realized as an x-ray CT imaging device as noted above, in other versions, the imaging system 104 may comprise one or more of an x-ray fluoroscopic imaging device, a magnetic resonance (MR) imaging device, a positron emission tomography (PET) imaging device, a single-photon emission computed tomography (SPECT), or an ultrasound imaging device. Other configurations are contemplated. In some versions, the imaging system 104 may be a mobile CT device that is not attached to the patient support 116 and may be wheeled or otherwise moved over the patient P and the patient support 116 to perform a scan. Examples of mobile CT devices include the BodyTom® CT scanner from Samsung Electronics Co., Ltd. and the O-arm® surgical imaging system form Medtronic, plc. The imaging system 104 may also be a C-arm x-ray fluoroscopy device. In other versions, the imaging system 104 may be a fixed-bore imaging device, and the patient P may be moved into the bore of the device, either on a patient support 116 or on a separate patient table that is configured to slide in and out of the central bore 112. Further, although the imaging system 104 shown in FIG. 1 is located close to the patient P within the operating room, the imaging system 104 may be located remotely, such as in another room or building (e.g., in a hospital radiology department).

The surgical system 100 employs the navigation system 102 to, among other things, track movement of various objects, such as the tools 106 and parts of the patient's P anatomy (e.g., tissue at the surgical site ST), as well as portions of the imaging system 104 in some versions. To this end, the navigation system 102 comprises a navigation controller 128 coupled to a localizer 130 that is configured to sense the position and/or orientation of trackers 132 within a localizer coordinate system LCLZ. In other words, the navigation system 102 includes the localizer 130 to track states of trackers 132 within a field of view. As is described in greater detail below, the trackers 132 (also referred to herein as “navigable trackers”) are fixed, secured, or otherwise attached to specific objects, and are configured to be monitored by the localizer 130.

The navigation controller 128 is disposed in communication with the localizer 130 and gathers position and/or orientation data for each tracker 132 sensed by the localizer 130 in the localizer coordinate system LCLZ. The navigation controller 128 may be disposed in communication with the imaging system controller 114 (e.g., to receive imaging data ID) and/or in communication with other components of the surgical system 100 (e.g., robotic arm controllers, tool controllers, and the like; not shown). However, other configurations are contemplated. The controllers 114, 128 may be realized as computers, processors, control units, and the like, and may be discrete components, may be integrated, and/or may otherwise share hardware.

It will be appreciated that the localizer 130 can sense the position and/or orientation of multiple trackers 132 to track correspondingly multiple objects within the localizer coordinate system LCLZ. By way of example, and as is depicted in FIG. 1, trackers 132 may comprise a tool tracker 132T, a pointer tracker 132P, an imaging system tracker 132I, one or more patient trackers 132A (e.g., a first patient tracker 132A, a second patient tracker 132B, and the like), a robot tracker 132R, as well as additional patient trackers, trackers for additional medical and/or surgical tools, and the like. The patient tracker 132A is adapted for attachment relative to the target site ST.

In FIG. 1, the tool tracker 132T, the pointer tracker 132P, and the imaging system tracker 132I are each depicted generically and are shown firmly fixed to (or otherwise integrated with) the rotary tool 108, the pointer tool 110, and the gantry 122 of the imaging system 104, respectively. The first and second patient trackers 132A, 132B, on the other hand, are removably coupled to mount assemblies 134 to define tracker assemblies 136 which facilitate selective movement of the trackers 132A, 132B relative to their mount assemblies 134 according to versions of the present disclosure, as described in greater detail below. As shown in FIG. 1, the tracker assemblies 136 are firmly fixed to different portions of the patient's P anatomy (e.g., to opposing lateral sides of the ilium) via anchors 138 which are configured to releasably engage tissue (e.g., bone). It will be appreciated that trackers 132 may be firmly affixed to different types of tracked objects (e.g., discrete bones, tools, pointers, and the like) in a number of different ways.

The position of the patient trackers 132A, 132B relative to the anatomy of the patient P to which they are attached can be determined by known registration techniques, such as point-based registration in which the pointer tool 110 (to which the pointer tracker 132P is fixed) is used to touch off on bony landmarks on bone, or to touch off on several points across the bone for surface-based registration. Conventional registration techniques can be employed to correlate the pose of the patient trackers 132A, 132B to the patient's anatomy. Other types of registration are also possible.

Position and/or orientation data may be gathered, determined, or otherwise handled by the navigation controller 128 using conventional registration/navigation techniques to determine coordinates of trackers 132 within the localizer coordinate system LCLZ. These coordinates may be utilized by various components of the surgical system 100 (e.g., to facilitate control of the tools 106, to facilitate navigation based on imaging data ID, and the like).

In the representative version illustrated in FIG. 1, the navigation controller 128 and the localizer 130 are supported on a mobile cart 140 which is movable relative to the base 120 of the imaging system 104. The mobile cart 140 also supports a user interface, generally indicated at 142, to facilitate operation of the navigation system 102 by displaying information to, and/or by receiving information from, the surgeon or another user. The user interface 142 may be disposed in communication with other components of the surgical system 100 (e.g., with the imaging system 104), and may comprise one or more output devices 144 (e.g., monitors, indicators, display screens, and the like) to present information to the surgeon (e.g., images, video, data, a graphics, navigable menus, and the like), and one or more input devices 146 (e.g., buttons, touch screens, keyboards, mice, gesture or voice-based input devices, and the like).

In some versions, the surgical system 100 is capable of displaying a virtual representation of the relative positions and orientations of tracked objects to the surgeon or other users of the surgical system 100, such as with images and/or graphical representations of the anatomy of the patient P and the tool 106 presented on one or more output devices 144 (e.g., a display screen). The navigation controller 128 may also utilize the user interface 142 to display instructions or request information from the surgeon or other users of the surgical system 100. Other configurations are contemplated. One type of mobile cart 140 and user interface 142 of this type of navigation system 102 is described in U.S. Pat. No. 7,725,162, entitled “Surgery System,” the disclosure of which is hereby incorporated by reference in its entirety.

Because the mobile cart 140 and the gantry 122 of the imaging system 104 can be positioned relative to each other and also relative to the patient P in the representative version illustrated in FIG. 1, the navigation system 102 can transform the coordinates of each tracker 132 from the localizer coordinate system LCLZ into other coordinate systems (e.g., defined by different trackers 132, localizers 130, and the like), or vice versa, so that navigation relative to the target site ST (or control of tools 106) can be based at least partially on the relative positions and orientations of multiple trackers 132 within a common coordinate system (e.g., the localizer coordinate system LCLZ). Coordinates can be transformed using a number of different conventional coordinate system transformation techniques. It will be appreciated that the localizer 130 or other components of the navigation system 102 could be arranged, supported, or otherwise configured in other ways without departing from the scope of the present disclosure. By way of non-limiting example, the localizer 130 could be coupled to the imaging system 104 in some versions (e.g., to the gantry 122). Other configurations are contemplated.

In the illustrated version, the localizer 130 is an optical localizer and includes a camera unit 148 with one or more optical position sensors 150. The navigation system 102 employs the optical position sensors 150 of the camera unit 148 to sense the position and/or orientation of the trackers 132 within the localizer coordinate system LCLZ. To this end, the trackers 132 each employ one or more markers 152 (also referred to as “fiducials” in some versions) that are supported on an array 154 in a predetermined arrangement. However, as will be appreciated from the subsequent description below, trackers 132 may have different configurations, such as with different quantities of markers 152 that can be secured to or otherwise formed in other structures besides the arrays 154 illustrated throughout the drawings (e.g., various types of housings, frames, surfaces, and the like). Other configurations are contemplated.

In some versions, certain trackers 132 (e.g., the patient tracker 132A) may employ “passive” markers 152 (e.g., reflective markers such as spheres, cones, and the like) which reflect emitted light that is sensed by the optical position sensors 150 of the camera unit 148. In some versions, trackers 132 employ “active” markers 152 (e.g., light emitting diodes “LEDs”), which emit light that is sensed by the optical position sensors 150 of the camera unit 148. Examples of navigation systems 102 of these types are described in U.S. Pat. No. 9,008,757, entitled “Navigation System Including Optical and Non-Optical Sensors,” the disclosure of which is hereby incorporated by reference in its entirety.

Although one version of the mobile cart 140 and localizer 130 of the navigation system 102 is illustrated in FIG. 1, it will be appreciated that the navigation system 102 may have any other suitable configuration for monitoring trackers 132 which, as will be appreciated from the subsequent description below, may be of various types and configurations and could employ various types of markers 152. Thus, for the purposes of clarity and consistency, the term “marker 152” is used herein to refer to a portion of a tracker 132 (e.g., a passive or active marker 152 mounted to an array 154 or otherwise coupled to a tracked object) that can be monitored by a localizer 130 to track (e.g., states, motion, position, orientation, and the like) of the object to which the tracker 132 is secured, irrespective of the specific type or configuration of the localizer 130 and/or tracker 132.

Those having ordinary skill in the art will appreciate that the navigation system 102 and/or localizer 130 may have any other suitable components or structure not specifically recited herein. Furthermore, any of the techniques, methods, and/or components described above with respect to the camera-based navigation system 102 shown throughout the drawings may be implemented or provided for any of the other versions of the navigation system 102 described herein. For example, the navigation system 102 may also be based on one or more of inertial tracking, ultrasonic tracking, image-based optical tracking (e.g., with markers 152 are defined by patterns, shapes, edges, and the like that can be monitored with a camera), or any combination of tracking techniques. Other configurations are contemplated.

As shown in FIG. 1, the surgical system 100 may include a robotic arm 156 operatively attached to a support element 158 and configured to maintain alignment of the tool 106 relative to the target site ST. The robotic arm 156 may extend between a base end 160 at the distal end 208 of the robotic arm 156 and a mount end 162 at the proximal end 210 arranged for movement relative to the base end 160. The surgical system 100 may further includes an end effector 164 attached to the mount end 162 of the robotic arm 156 and configured to support one or more types of tools 106, instruments, and the like. More specifically, the surgical system 100 may further include a tool guide 166 supported by the end effector 164, and the tool guide 166 may be configured to support the tool 106 relative to a trajectory that is aligned or otherwise determined relative to the surgical site ST on the patient P.

The robotic arm 156 may comprise a multi-joint arm that includes a plurality of linkages connected by joints having actuator(s) and optional encoder(s) (not shown in detail) to enable the linkages to bend, rotate and/or translate relative to one another in response to control signals from a robot control system. In other words, the robotic arm moves in a plurality of degrees of freedom. In some examples, the robotic arm moves in at least three degrees of freedom. In other examples, the robotic arm is configured to move in at least three degrees of freedom. In some examples, such as shown in FIG. 1, the robotic arm 156 may be fixed to the imaging system 104, such as on the support element 158 (e.g., a curved rail) that may extend concentrically over the outer surface of the O-shaped imaging gantry 122 of the imaging system 104 and that may be located close to the target site ST of the patient P. In some versions, the robotic arm 156 could be coupled to a mobile cart (not shown) or to another type of support element 158 that is not necessarily coupled to the imaging system 104, such as described in U.S. Pat. No. 10,603,119, entitled “Steering Mechanism for Portable Surgical Robot,” the disclosure of which is hereby incorporated by reference in its entirety. Although a single robotic arm 156 is shown in FIG. 1, it will be understood that the surgical system 100 may include multiple robotic arms attached to suitable support structure(s). Other configurations are contemplated.

The support element 158 may form a semicircular arc and may be concentric with the outer circumference of the O-shaped imaging gantry 122. The support element 158 may extend around at least 25%, such as between about 30-50% of the outer circumference of the O-shaped imaging gantry 122. The support element 158 may extend around at least a portion of the outer circumference of the O-shaped imaging gantry 122 that is located above the target site ST of the patient P. More specifically, the base end 160 of the robotic arm 156 (e.g., the end of the robotic arm 156 opposite the end effector 164) may be fixed to the support element 158, in a non-limiting example, at a position that is less than about 2 meters, such as less than about 1 meter (e.g., between 0.5 and 1 meter) from the surgical site ST of the patient P during a surgical procedure.

In versions, the support element 158 may extend along a semicircular arc having a radius that is greater than about 33 inches, such as greater than about 35 inches (e.g., between 33 and 50 inches). The support element 158 may be spaced from the outer surface of the O-shaped imaging gantry 122 by a pre-determined distance, which may be from less than an inch (e.g., 0.5 inches) to 6 or 10 inches or more. In some versions, the support element 158 may be spaced from the O-shaped imaging gantry 122 by an amount sufficient to enable the tilt motion of the O-shaped imaging gantry 122 with respect to a gimbal 168 supporting the O-shaped imaging gantry 122 over at least a limited range of motion. Additionally, in some versions, the support element 158 may comprise one or more straight segments (e.g., rail segments), where at least a portion of the support element 158 may extend over the top surface of the O-shaped imaging gantry 122. Other configurations are contemplated.

A carriage 170 may be located on the support element 158 and may include a mounting surface 172 for mounting the base end 160 of the robotic arm 156 to the carriage 170. As shown in FIG. 1, the carriage 170 may extend from the support element 158 towards a first (e.g., front) face of the O-shaped imaging gantry 122. The mounting surface 172 for the robotic arm 156 may extend beyond the first (e.g., front) face of the O-shaped imaging gantry 122 and the robotic arm 156 may extend over the first (e.g., front) face of the O-shaped imaging gantry 122. In some versions, the configuration of the carriage 170 and the mounting surface 172 may be reversed such that the mounting surface 172 extends beyond the second (e.g., rear) face of the O-shaped imaging gantry 122, and the robotic arm 156 may extend over the second (e.g., rear) face of the O-shaped imaging gantry 122. In this configuration, the patient support 116 may be configured such that the patient support 116 and patient P extend into or through the central bore 112 defined through the O-shaped imaging gantry 122, and a portion of the patient P requiring surgical intervention (e.g., the cranium) may be accessed from the second (e.g., rear) side of the O-shaped imaging gantry 122.

In some versions, the carriage 170 and the robotic arm 156 attached thereto may be moved to different positions along the length of support element 158 (e.g., any arbitrary position between a first end 176 and a second end 178 of the support element 158). The carriage 170 and the robotic arm 156 may be fixed in place at a particular desired position along the length of the support element 158. In some versions, the carriage 170 may be moved manually (e.g., positioned by an operator at a particular location along the length of the support element 158 and then clamped or otherwise fastened in place). Alternately, the carriage 170 may be driven to different positions using a suitable drive mechanism (e.g., a motorized belt drive, friction wheel, gear tooth assembly, cable-pulley system, etc., not shown in detail). The drive mechanism may be located on the carriage 170 and/or the support element 158, for example. An encoder mechanism may be utilized to indicate the position of the carriage 170 and the base end 160 of the robotic arm 156 on the support element 158. Although the version of FIG. 1 illustrates one robotic arm 156 mounted to the support element 158, it will be understood that more than one robotic arm 156 may be mounted to the support element 158 via respective carriages 170.

In some versions, the robotic arm 156 may be mounted directly to the support element 158, such as on a mounting surface 172 that is integrally formed on the support element 158. In such an version, the position of robotic arm 156 may not be movable along the length of the support element 158. In other versions, the robotic arm 156 may be secured to any other portion of the imaging system 104, such as directly mounted to the gantry 122. Alternatively, the robotic arm 156 may be mounted to the patient support 116 or column 118, to any of the wall, ceiling or floor in the operating room, or to a separate cart as noted above. In some versions, the robotic arm 156 may be mounted to a separate mobile shuttle, similar to as is described in U.S. Pat. No. 11,103,990, entitled “System and Method for Mounting a Robotic Arm in a Surgical Robotic System,” the disclosure of which is hereby incorporated by reference in its entirety. Although a single robotic arm 156 is shown in FIG. 1, it will be understood that two or more robotic arms 156 may be utilized.

Those having ordinary skill in the art will appreciate that the robotic arm 156 can be employed to aid in the performance of various types of surgical procedures, such as a minimally-invasive spinal surgical procedure or various other types of orthopedic, neurological, cardiothoracic and general surgical procedures. In the version of FIG. 1, the robotic arm 156 may be used to assist a surgeon performing a surgical procedure in the lumbar spinal region of a patient. The robotic arm 156 may also be used for thoracic and/or cervical spinal procedures. The procedures may be performed posteriorly, anteriorly or laterally. Other configurations are contemplated.

In some versions, the robotic arm 156 may be controlled to move the end effector 164 to one or more pre-determined positions and/or orientations with respect to a patient P, such as to and/or along a trajectory defined relative to the anatomy of the patient P. As discussed above, the end effector 164 may be realized as or may otherwise support various types of instruments and/or tools 106 including, but not limited to, a needle, a cannula, a dilator, a cutting or gripping instrument, a scalpel, a drill, a screw, a screwdriver, an electrode, an endoscope, an implant, a radiation source, a drug, etc., that may be inserted into the body of the patient P. In some versions, the end effector 164 may be realized as a hollow tube or cannula configured to receive a surgical tool 106, including without limitation a needle, a cannula, a dilator, a cutting or gripping instrument, a scalpel, a drill, a screw, a screwdriver, an electrode, an endoscope, an implant, a radiation source, a drug, and the like. The surgical tool 106 may be inserted into or otherwise adjacent to the patient's body through the hollow tube or cannula by a surgeon. The robotic arm 156 may be controlled to maintain the position and orientation of the end effector 164 with respect to the patient P to ensure that the surgical tool(s) 106 follow a desired trajectory through the patient's body to reach the target site ST. The target site ST may be determined preoperatively and/or intraoperatively, such as during a surgical planning process, based on patient images which may be obtained using the imaging system 104.

In the representative version illustrated herein, the navigation system 102 tracks the robotic arm 156 within the localizer coordinate system LCLZ via the robot tracker 132R, as is described in greater detail below. To this end, a control loop may continuously read the tracking data and current parameters (e.g., joint parameters) of the robotic arm 156, and may send instructions to the navigation controller 128 and/or to the imaging system controller 114 (and/or some other controller, such as a robot controller) to cause the robotic arm 156 to move to a desired position and orientation within the localizer coordinate system LCLZ.

In some versions, a surgeon may use one or more portions of the surgical system 100 as a planning tool for a surgical procedure, such as by setting trajectories within the patient for inserting tools 106, as well as by selecting one or more target sites ST for a surgical intervention within the patient's body. The trajectories and/or target sites ST set by the surgeon may be saved (e.g., in a memory of a computer device) for later use during surgery. In some versions, the surgeon may be able to select stored trajectories and/or target sites ST using the surgical system 100, and the robotic arm 156 may be controlled to perform a particular movement based on the selected trajectory and/or target site ST. For example, the robotic arm 156 may be moved to position the end effector 164 of the robotic arm 156 into alignment with the pre-defined trajectory and/or over the pre-determined target site ST. As discussed above, the end effector 164 may include the tool guide 166 which may be used to guide the tool 106 relative to the patient's body along the pre-defined trajectory and/or to the pre-defined target site ST.

As discussed above, the localizer 130 may include a camera unit 148 with one or more optical position sensors 150. More specifically, the optical position sensors 150 may be light sensors capable of sensing changes in infrared (IR) emitted within a field of view. In some versions, the localizer 130 may include one or more radiation sources (e.g., one or more diode rings) that direct radiation (e.g., IR radiation) into the surgical field, where the radiation may be reflected by the markers 152 and received by the cameras. In the illustrated version, certain active markers 152 (e.g., active markers 152 which define the robot tracker 132R) are configured to emit IR light detectable by the optical position sensors 150 of the localizer 130. The navigation controller 128 may be coupled to the localizer 130 and may determine the positions and/or orientations of markers 152 detected by the optical position sensors 150 using, for example, triangulation and/or transformation techniques. A 3D model and/or mathematical simulation of the surgical space may be generated and continually updated using motion tracking software implemented by the navigation controller 128.

As discussed above, the patient tracker 132A may be rigidly attached to a portion of the patient's anatomy in the anatomical region of interest adjacent to the target site ST (e.g., clamped or otherwise attached to the ilium, to the spinous process of the vertebrae, and the like) to enable the anatomical region of interest to be continually tracked by the navigation system 102. In the illustrated version, the robot tracker 132R includes an end effector tracker 182 that is rigidly attached to the end effector 164 of the robotic arm 156 to enable the robotic arm 156 to be tracked using the navigation system 102. Using the pose of the end effector tracker 182 (as well as of the patient tracker 132) monitored within the localizer coordinate system LCLZ by the localizer 130, the navigation controller 128 and/or some other controller (e.g., a robot controller) may include software configured to perform transformations between joint coordinates of the robotic arm 156 and the localizer coordinate system LCLZ which, in turned, may be utilized by the robotic arm 156 to control or otherwise adjust the position and/or orientation of the end effector 164 with respect to the patient P. In some versions, the robotic arm 156 may include multiple robot trackers 132R and/or robot trackers 132R other than the end effector tracker 182 (e.g., on joints of the arm). Other configurations are contemplated.

In the illustrated version, the end effector 164 includes a plurality of LED markers 184. Each LED marker 184 includes a respective light module (e.g., LED) arranged to emit light. Each LED marker 184 further includes a lens 198 to diffuse light emitted by the light module. The lens 198 is arranged to present diffused light across the LED marker 184 detectable by the localizer 130 of the navigation system 102 to enable tracking states of the end effector 164.

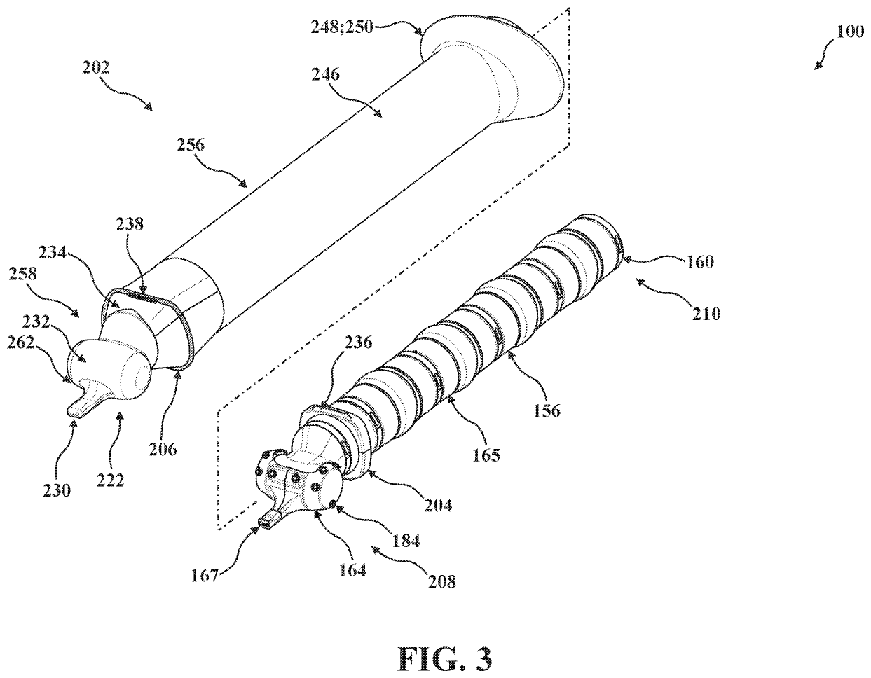

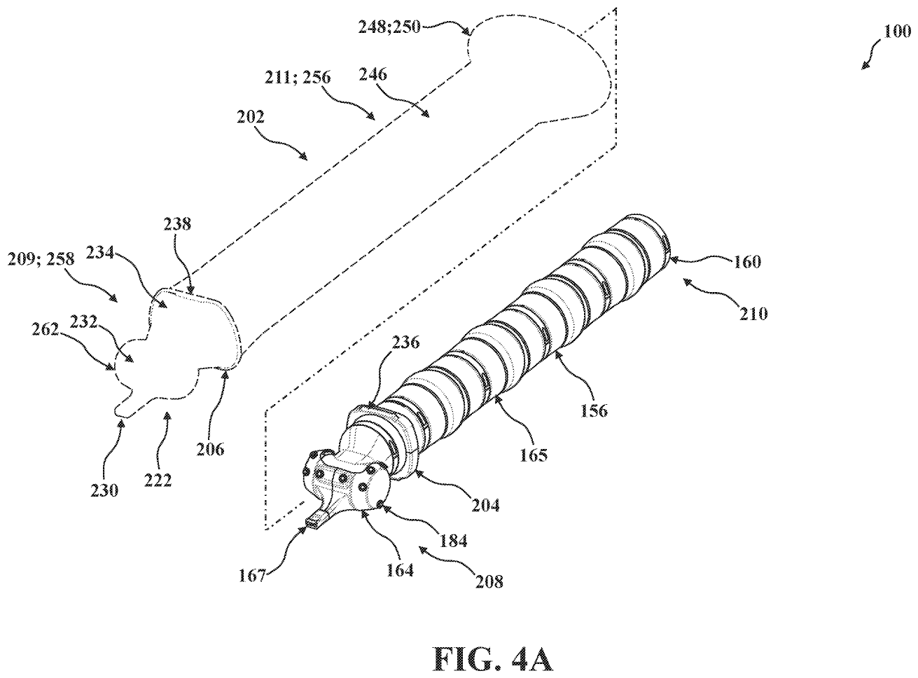

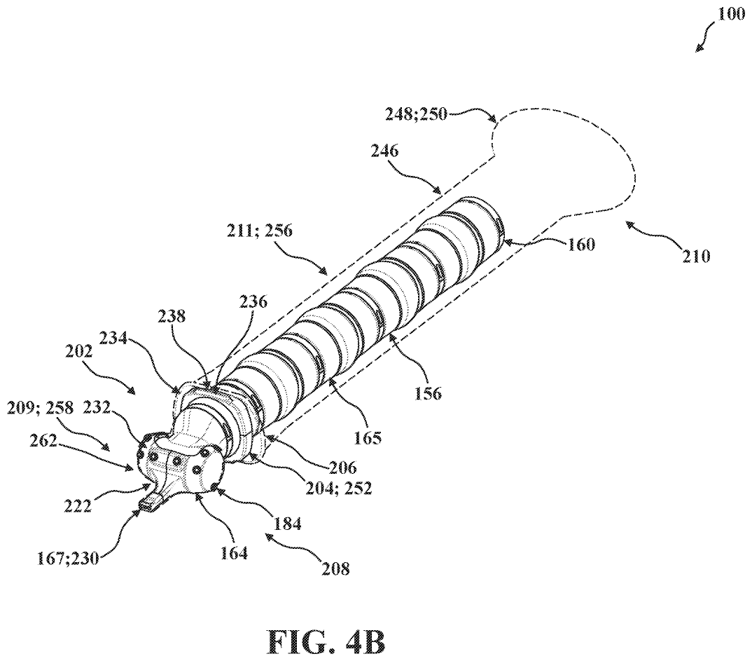

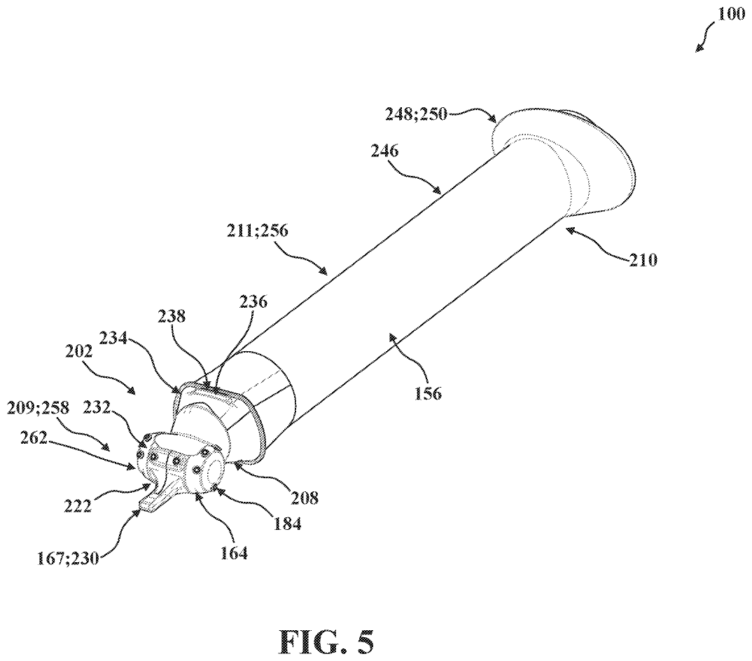

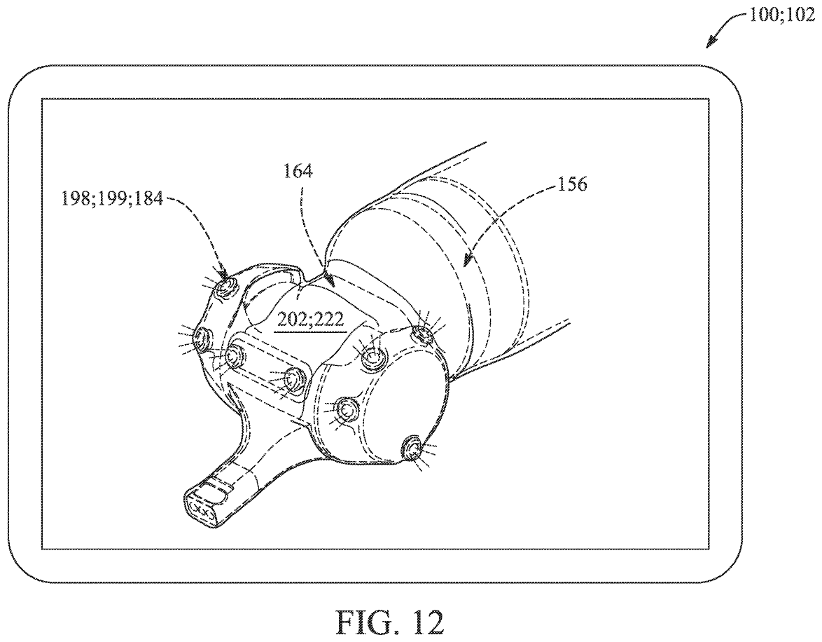

Referring now to FIGS. 3-5, portions of a surgical system 100 according to aspects of the present disclosure is shown. The surgical system 100 generally includes a robotic arm 156 with a plurality of links 165 extending between an end effector 164 and the base end 160. The end effector 164 supports a plurality of LED markers 184 arranged for observation by a navigation system 102 (see FIGS. 1 and 12;) based on optical sensing technology. The end effector 164 also defines or otherwise supports a tool mount 167 extending proximally and configured to releasably secure one or more types of surgical tools, instruments, and the like (described above and generally known in the art). The robotic arm 156 also includes a keeper 204 disposed on one of the links 165 adjacent to the end effector 164. As is described in greater detail below, the keeper 204 is employed to facilitate releasably securing a drape assembly 202 to the robotic arm 156 to cover portions of the robotic arm 156 for sterility in connection with various types of surgical procedures.

The robotic arm 156 can be mounted or otherwise supported in various ways via the base end 160, and is generally configured to facilitate guiding, moving, or otherwise aligning the end effector 164 relative to a target site (e.g., of a patient's anatomy) via operation of the links 165. In some versions, aspects of the robotic arm 156, the navigation system 102, or other portions of the surgical system 100 may be similar to as is disclosed in one or more of: U.S. Pat. No. 10,959,783 entitled “Integrated Medical Imaging and Surgical Robotic System,” U.S. Pat. No. 10,653,495 entitled “Methods and Systems for Display of Patient Data in Computer-Assisted Surgery,” U.S. Pat. No. 11,103,990 entitled “System and Method for Mounting a Robotic Arm in a Surgical Robotic System,” U.S. Pat. No. 10,828,112 entitled “Methods and Systems for Setting Trajectories and Target Locations for Image Guided Surgery,” U.S. Patent Application Publication No. 2020/0078097 entitled “Methods and Systems for Robot-Assisted Surgery,” and U.S. Patent Application Publication No. 2021/0236207 entitled “Methods and Systems for Controlling a Surgical Robot,” the disclosures of each of which are hereby incorporated by reference in their entirety. Other configurations are contemplated.

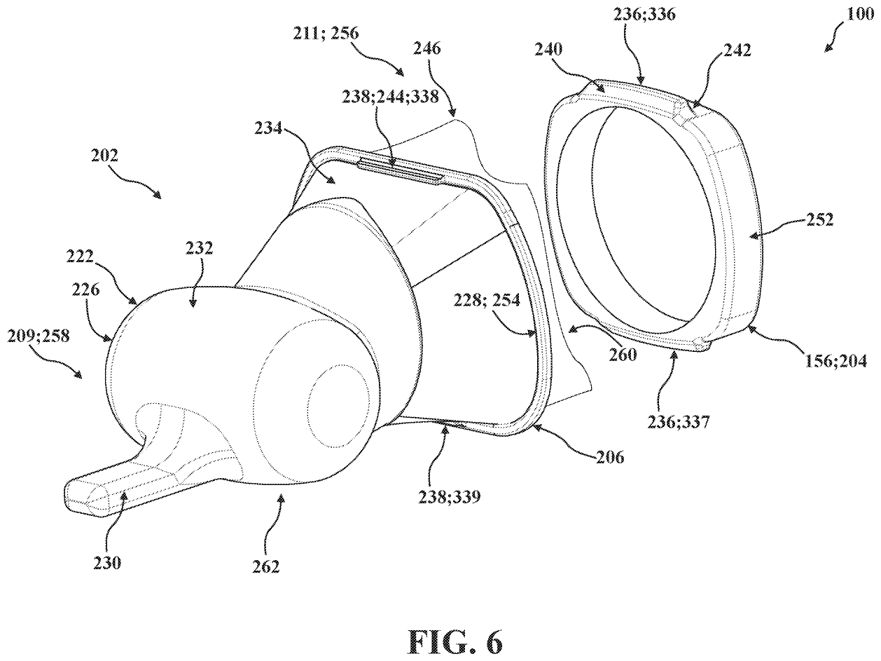

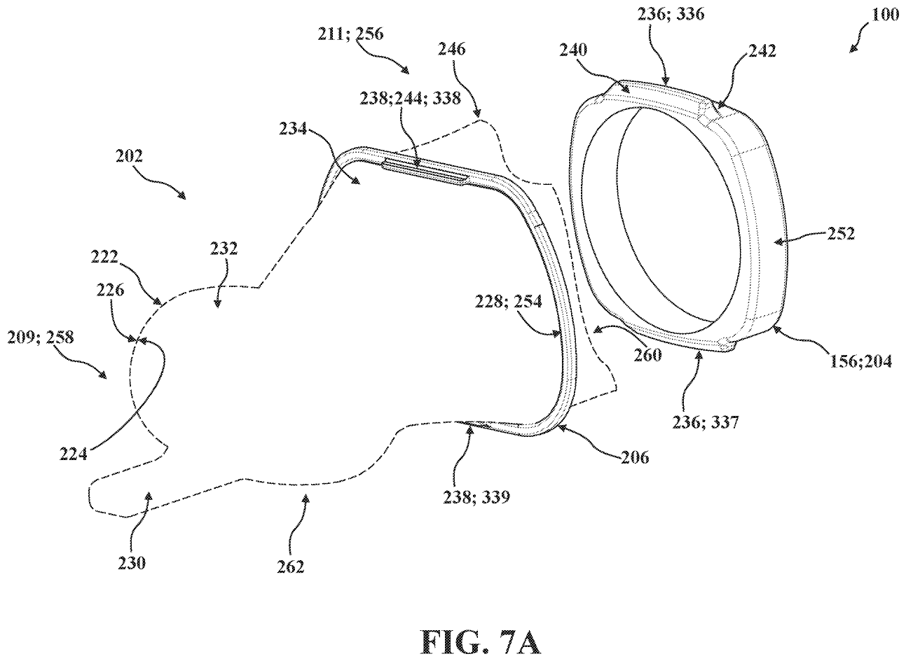

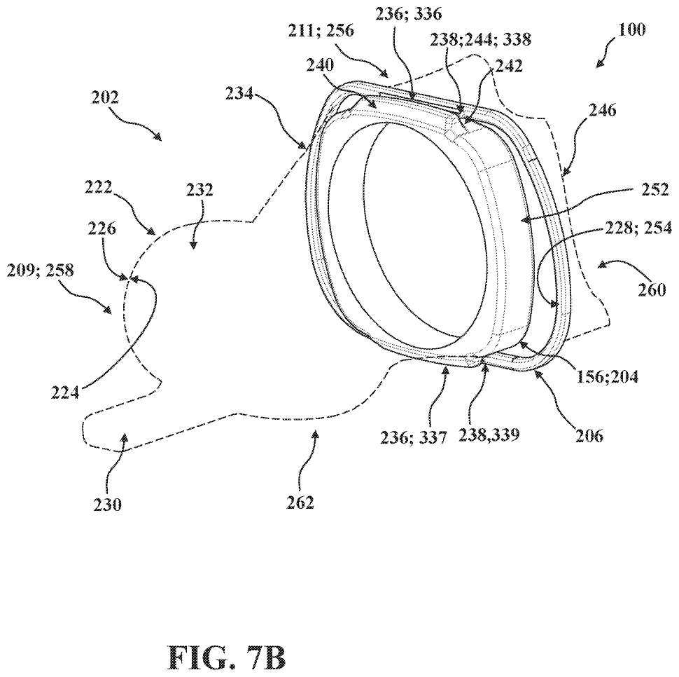

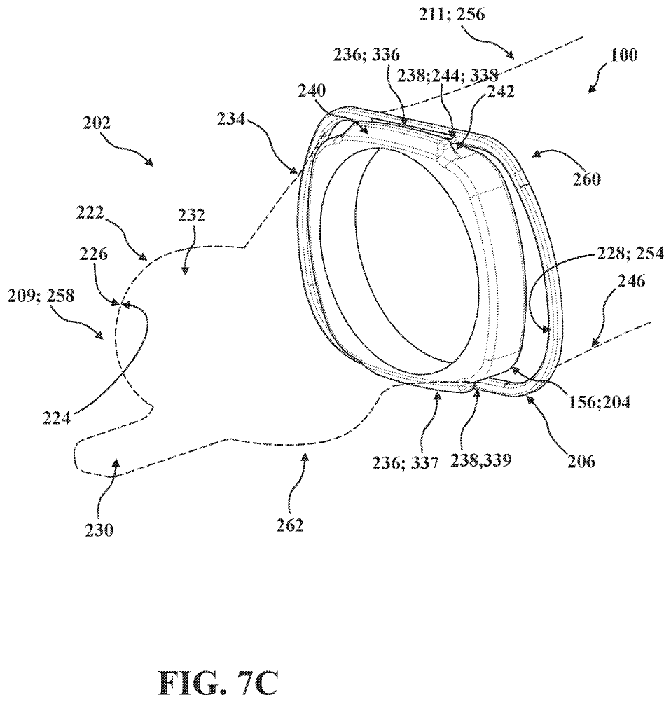

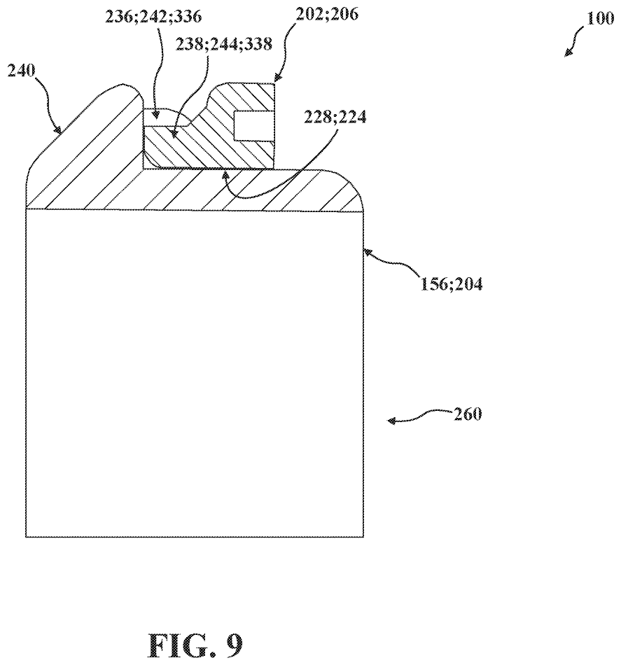

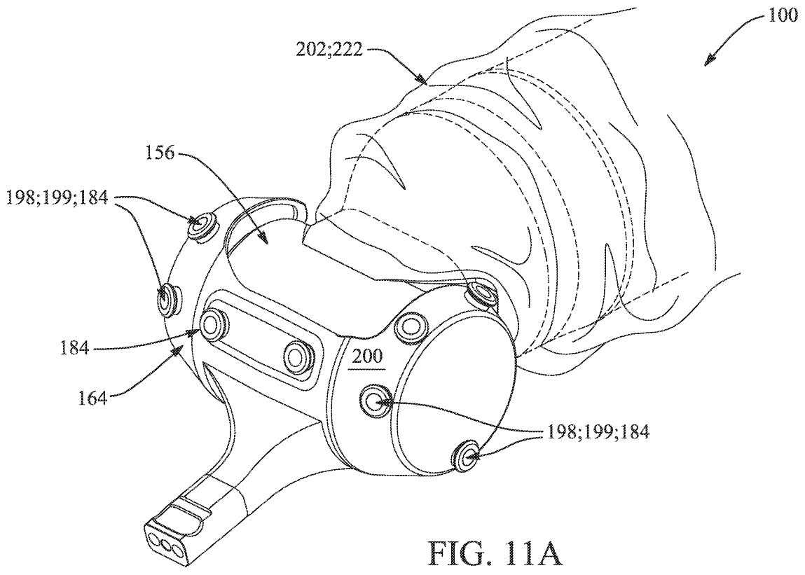

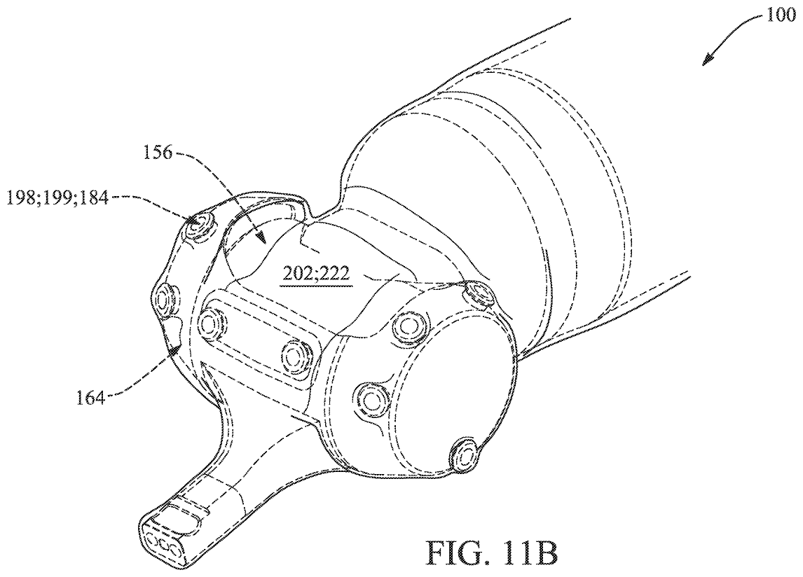

Referring now, generally, to FIGS. 3-12, the drape assembly 202 is configured for releasable attachment to the robotic arm 156 via the keeper 204 mounted adjacent to the end effector 164, as noted above. The resilient drape 222 comprises a first portion 256 defining an opening 260 shaped to receive the distal end 208 of the robotic arm 156 therethrough and a second portion 258 in fluid communication with the first portion 256 and defining a cavity 262 shaped to accommodate at least a portion of the distal end 208 of the robotic arm 156 therein. The cavity 262 of the resilient drape 222 includes a mount region 230 which corresponds to where the tool guide 166 mounts, a body region 232 which corresponds to the end effector 164, and a tapered region 234 which transitions the resilient drape 222 between the first portion 256 and the second portion 258 leading to the opening 260. To this end, the drape assembly 202 generally includes a guide 206 to which a resilient drape 222 is coupled. The resilient drape 222 is pre-formed with a shape that substantially corresponds to the end effector 164 of the robotic arm 156 and is sized and configured to as to “stretch” over the end effector 164 and remain secured thereto via engagement between the guide 206 and the keeper 204, as described in greater detail below. The resilient drape 222 is manufactured from a conformable, resilient material which permits light emitted by the LED markers 184 (e.g., infrared light, visible light, and the like) to be detected by the navigation system 102. Put differently, the resilient drape 222 is configured so as to be transmissive to light emitted by the LEDs 184 and conforms to the end effector 164 in a way which does not present undesirable folds, creases, improper diffusion, and the like, which would otherwise inhibit visibility of the LED markers 184 via the navigation system 102. In the representative versions depicted herein, the resilient drape 222 is manufactured from polyisoprene. However, it will be appreciated that other suitable types of resilient, conformable, light transmissive materials are contemplated by the present disclosure.

The resilient drape 222 may include several material characteristics. The resilient drape 222 may have a tensile strength of about 10 MPa to about 30 MPa. In some examples, the tensile strength may be between 15 MPa and 20 MPa. The resilient drape 222 may have an elongation of between about 500 percent and about 1000 percent. In some examples, the elongation may be 750 percent or more. The resilient drape 222 may have a 100 percent modulus between about 0.7 MPa and about 1.5 MPa. The 100 percent modulus may be 1.0 MPa. The resilient drape 222 may have a 300 percent modulus of about 1.0 MPa to about 2.0 MPa. The 300 percent modulus may be 1.6 MPa. The resilient drape 222 may have a hardness of 36 Shore A (ASTM D 2240). The resilient drape 222 may have a specific gravity between about 0.75 grams/cc and about 1.25 grams/cc. The specific gravity may be 0.90 grams/cc to 1.0 grams/cc. Other characteristics of the resilient drape material consistent with the above described features are also contemplated.

The resilient drape 222 is configured to cover and contact the LED markers 184 on the outer surface 200 of the end effector 164 such that diffused light presented across the lens 198 can be viewed by the localizer 130 with a consistent and substantially uniform intensity, from various angles and perspectives, including when viewed directly on. Those having ordinary skill in the art will appreciate that the consistent intensity of diffused light afforded by the LED markers 184 through the resilient drape 222 of the present disclosure provides significant advantages to improved tracking accuracy and reliability by preventing the LED markers 184 from being distorted or lost by the navigation system 102 markers with inconsistently bright and dull intensity (which, thus, would otherwise complicate accurate monitoring of other markers). Thus, the localizer 130 is able to reliably detect light emitted from each of the LED markers 184 that are visible within its field of view, as well as from other trackers 132 (e.g., the patient tracker 132A) that are visible within its field of view, with a high level of accuracy irrespective of movement of markers 184. Accordingly, the navigation system 102 can accurately monitor for relative movement between the patient and the robotic arm 156 during the surgical procedure which, in turn, also allows the robotic arm 156 and other portions of the surgical system 100 to react to movement of tracked objects.

Furthermore, it will be appreciated that the resilient drape 222 functions as a filter for the LED markers 184 of the end effector 164, which helps ensure that the localizer 130 can accurately locate each of the LED markers 184 irrespective of its orientation. More specifically, the uniformity and consistency of the light presented across the lenses 198 help prevent navigation inaccuracy issues that could otherwise occur when viewing light emitting diodes 199 which emit light that varies in intensity depending on the orientation it is viewed from. In this example, which utilizes polyisoprene, the resilient drape 222 allows the infrared and visible light to freely pass through the material without distortion to the localizer 130 of the navigation system 102. In some examples, the resilient drape 222 may have a thickness of about 0.004 inches to about 0.02 inches. In some examples, the resilient drape 222 may have a first thickness at mount region 230 and a second thickness at the opening 260. The thickness of the resilient drape 222 may taper from the mount region 230 to the opening 260. In some examples, the first thickness may be 0.012 inches and the second thickness may be 0.006 inches. In some examples, the resilient drape 222 may have a uniform thickness between the mount region 230 and the opening 260.

Furthermore, when the LED markers 184 were tested with alternative materials (e.g., latex; polypropylene; the like), there was a notable reduction in transmission between the LED markers 184 and the signals received by the localizer 130 when the end effector 164 was moved in a plurality of degrees of freedom. Here too, it will be appreciated that the consistent intensity of diffused light presented across the LED markers 184 allows the end effector 164 to be known and readily recognized, even where the end effector 164 is arranged at angles relative to the field of view of the localizer 130 that would otherwise complicate recognizing a known or otherwise expected shape and/or profile. In this way, potential tracking issues resulting from optical distortions present when directly monitoring certain types of light emitting diodes 199, can be significantly minimized.

The resilient drape 222 is considered a sterile object and generally includes an inner drape surface 224 and an outer drape surface 226. The inner drape surface 224 is configured to engaged against portions of the end effector 164 and the LEDs 184, which may not separately be considered sterile objects. The outer drape surface 226 of the resilient drape 222 maintains sterility and helps to define the sterile barrier with the portions of the robotic arm 156 enclosed by the resilient drape 222. The guide 206 of the drape assembly 202 is operatively attached to the resilient drape 222 and is configured to facilitate optimized handling of the drape assembly 202 to releasably secure to the robotic arm 156 via the keeper 204. Here, the guide 206 is realized with a tapered, oblong, “ring-shaped” profile and is formed from a relatively rigid, resilient material (e.g., plastic). The guide 206 defines a guide aperture 228 arranged in communication with the inner drape surface 224 of the resilient drape 222 and is shaped and arranged to be grasped by a user (e.g., a caregiver, technician, nurse, and the like) during attachment to the robotic arm 156. Here, it will be appreciated that the guide aperture 228 and the guide 206 itself are sized larger than the end effector 164 to enable the user to guide the resilient drape 222 onto and over the end effector 164 without touching the end effector 164 or other portions of the robotic arm 156, and also without touching the inner drape surface 224 of the resilient drape 222. Put differently, this configuration allows the user to attach the guide 206 to the keeper 204 without touching non-sterile components of the robotic arm 156.

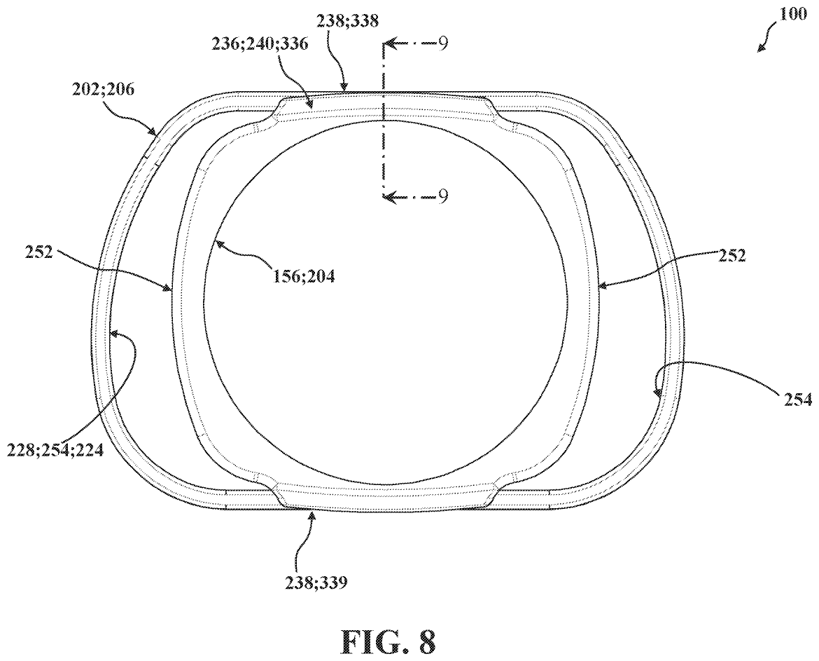

The resilient drape 222 is shaped so as to correspond to the end effector 164 as noted above, and the illustrated versions generally include a mount region 230 shaped to receive the tool mount 167 of the end effector 164, a body region 232 shaped to receive the portion of the end effector 164 which supports the LEDs 184, and a tapered region 234 extending from the body region 232 to the guide 206. In use, the mount region 230 and the body region 232 remain in relatively close contact with the end effector 164, and the tapered region 234 may be configured to hold close contact with the end effector 164 adjacent to the body region 232 but may extend out of contact with the robotic arm 156 for airflow or cooling purposes. Here, for example, because the guide aperture 228 is sized larger than the outer profile 252 of the keeper 204 (see FIG. 8), airflow may be directed along the tapered region 234 relative to other portions of the robotic arm 156 in some versions. For example, in FIG. 8, channels are formed between the inner perimeter 254 of the guide aperture 228 and the outer profile 252 of the keeper 204 allowing airflow to pass between the tapered region 234 near the distal end of the robotic arm 156 and the proximal end 210 of the robotic arm 156, along the length of the drape assembly 202 ending at the cinch 250.

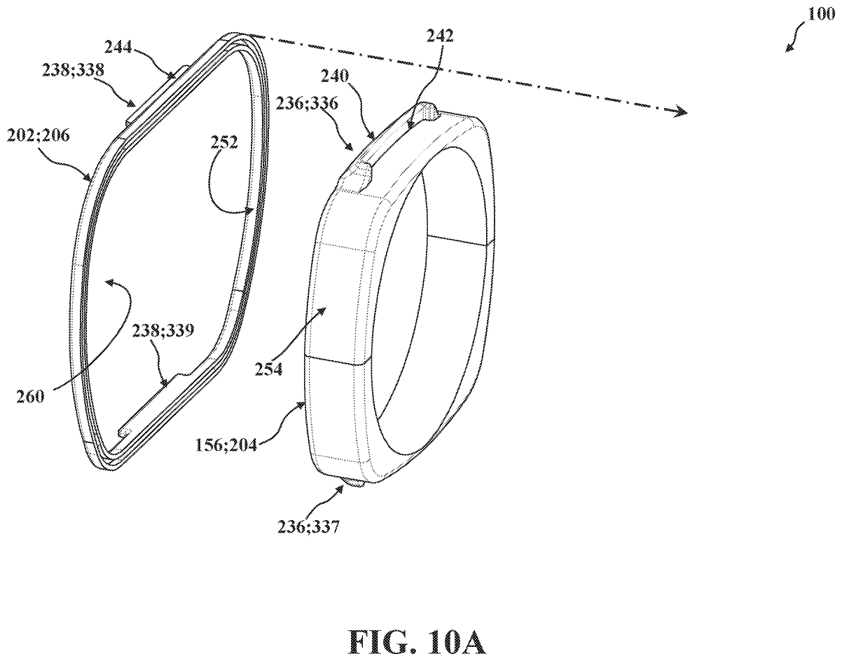

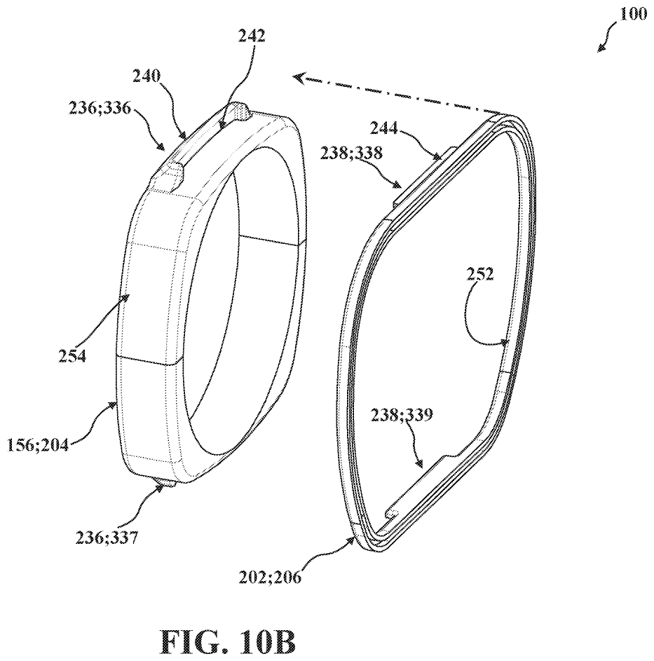

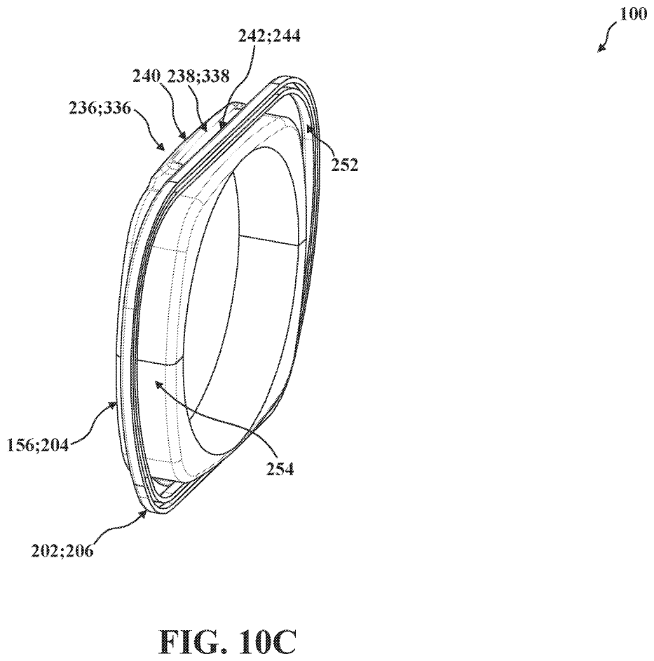

Referring now to FIGS. 8-10C, in order to facilitate releasable engagement of the drape assembly 202 to the robotic arm 156, the keeper 204 has a generally rigid profile that is configured to be operatively attached to the robotic arm 156 (e.g., secured to one or the links 165, formed integrally with the robotic arm 156, and the like), and defines one or more keeper interfaces 236, 336, 337 that interact with one or more corresponding guide interfaces 238, 338, 339 of the guide 206. Here, the keeper interfaces 236, 336, 337 have keeper tapers 240 arranged so as to face generally away from the base end 160 and towards the end effector 164, and keeper seats 242 which generally face away from the end effector 164. The keeper interfaces 236 may be a first keeper interface 336 and a second keeper interface 337. The guide interfaces 238 may be a first guide interface 338 and a second guide interface 339. Each set of the first guide interface 238, 338 and the second guide interface 238, 339 may releasably engage one of the first keeper interface 236, 336 and the second keeper interface 236, 337. The keeper tapers 240 are configured with ramped profiles which facilitate deflecting the guide 206 to temporarily “enlarge” the guide aperture 228 in response to force exerted on the guide 206 towards proximally by the user during assembly (e.g., as is illustrated in exaggerated sequence from FIG. 10A to FIG. 10B). While not depicted in detail, the resiliency of the guide 206 allows it to pass over the keeper tapers 240 to become positioned distally of the keeper 204 (as is depicted in exaggerated fashion in FIG. 10B) and then the retainers 244 of the guide 206 can be positioned into the keeper seats 242 to retain the guide 206 to the keeper 204 (as is depicted in FIG. 10C). It will be appreciated that various types, styles, and arrangements of retainers 244, keeper seats 242, and/or keeper tapers 240 may be utilized without departing from the scope of the present disclosure.

In the representative version illustrated herein, the drape assembly 202 also includes a tube drape 246 operatively attached to the guide 206 distal of the resilient drape 222. Here, the tube drape 246 may be configured from the same material as the resilient drape 222 or may be manufactured from some other suitable material such as another type of plastic. In some examples, the plastic material may be polyethylene, polypropylene, polystyrene, polycarbonate, polyethylene terephthalate glycol (PETG), polyetheretherketone (PEEK), polyester, polypropylene, polyethylene or polytetrafluoroethylene (PTFE) or the like. In some versions, the tube drape 246 may be arranged to cover additional portions of the robotic arm 156 (e.g., distal links 165), and may extend to a billowed portion 248 which is shaped to be positioned over other portions of the robotic arm 156, such as to a cooling system (not shown) configured to circulate air along the robotic arm 156 and through the tube drape 246, as well as through the guide aperture 228 of the guide 206 and partially into the tapered region 234 of the resilient drape 222. A cinch 250 may be provided adjacent to the billowed portion 248 to facilitate attachment to the robotic arm 156 or other portions of the surgical system 100.

It will be further appreciated that the terms “include,” “includes,” and “including” have the same meaning as the terms “comprise,” “comprises,” and “comprising.”

Several versions have been discussed in the foregoing description. However, the versions discussed herein are not intended to be exhaustive or limit the invention to any particular form. The terminology which has been used is intended to be in the nature of words of description rather than of limitation. Many modifications and variations are possible in light of the above teachings and the embodiments may be practiced otherwise than as specifically described. Moreover, two or more of the versions disclosed herein may be combined with or without modification.

The present disclosure also comprises the following clauses, with specific features laid out in dependent clauses, that may specifically be implemented as described in greater detail with reference to the configurations and drawings above.

### Clauses

I. A robotic surgical system comprising:
a robotic arm extending between a distal end and a proximal end;
a keeper coupled to the robotic arm, the keeper having a keeper interface; and
a drape including:
   a first portion defining an opening shaped to receive the distal end of the robotic arm therethrough,
   a second portion disposed in fluid communication with the first portion and defining a cavity shaped to accommodate at least a portion of the distal end of the robotic arm therein, and
   a guide disposed between the first portion and the second portion and having a guide interface configured to releasably attach to the keeper interface of the keeper to secure the drape to the robotic arm.

II. The robotic surgical system of clause I, wherein the keeper interface of the keeper includes a seat; and
wherein the guide includes a retainer operatively attached to the guide and shaped to releasably engage the seat of the keeper.

III. The robotic surgical system of any of clauses I-II, wherein the guide is at least partially formed from a resilient material.

IV. The robotic surgical system of clause III, wherein the keeper interface includes a taper having a ramped profile shaped to resiliently enlarge the guide as the guide is passed over the keeper.

V. The robotic surgical system of any of clauses I-IV, wherein the guide defines an aperture having an inner perimeter larger than an outer profile of the keeper.

VI. The robotic surgical system of clause V, wherein the guide defines an airflow channel arranged between the outer profile of the keeper and the inner perimeter of the aperture of the guide.

VII. The robotic surgical system of any of clauses I-VI, further comprising an end effector operatively attached to the distal end of the robotic arm, the end effector including a plurality of markers protruding from a surface of the end effector; and
wherein the second portion of the drape is formed from an elastic polyisoprene material shaped to cover the end effector and contact each of the plurality of markers.

VIII. The robotic surgical system of clause VII, further comprising a navigation system configured to optically track the plurality of markers on the end effector through the second portion of the drape.

IX. The robotic surgical system of any of clauses I-VIII, wherein the first portion of the drape and the second portion of the drape are formed from different materials.

X. The robotic surgical system of clause IX, wherein the second portion of the drape is formed from an elastic polyisoprene material.

XI. The robotic surgical system of any of clauses I-VIII, wherein the first portion of the drape and the second portion of the drape are formed from the same material.

XII. The robotic surgical system of clause XI, wherein the second portion of the drape is formed from an elastic polyisoprene material.

XIII. The robotic surgical system of any of clauses I-XII, wherein the first portion of the drape includes:
a distal section coupled to the guide, and
a proximal section defining the opening.

XIV. The robotic surgical system of any of clauses I-XIII, wherein the keeper includes two keeper interfaces; and
wherein the guide includes two guide interfaces to respectively releasably attach to the two keeper interfaces.

XV. The robotic surgical system of any of clauses I-XIV, wherein the keeper is integrated into the robotic arm adjacent to the distal end.

XVI. The robotic surgical system of any of clauses I-XIV, wherein the keeper is removably connected to the robotic arm adjacent to the distal end.

XVII. A drape assembly for a robotic arm of a robotic surgical system, the drape assembly comprising:
a keeper for securing to the robotic arm, the keeper having a keeper interface; and
a drape including:
   a first portion defining an opening shaped to receive a distal end of the robotic arm therethrough,
   a second portion disposed in fluid communication with the first portion and defining a cavity shaped to accommodate at least a portion of the distal end of the robotic arm therein, and
   a guide disposed between the first portion and the second portion and having a guide interface configured to releasably attach to the keeper interface of the keeper to secure the drape to the robotic arm.

XVIII. The drape assembly of clause XVII, wherein the keeper interface of the keeper includes a seat; and
wherein the guide includes a retainer operatively attached to the guide and shaped to releasably engage the seat of the keeper.

XIX. The drape assembly of any of clauses XVII-XVIII, wherein the guide includes a first guide interface and a second guide interface, the first guide interface being spaced from the second guide interface.

XX. The drape assembly of clause XIX, wherein the keeper includes a first keeper interface and a second keeper interface, the first keeper interface being spaced from the second keeper interface.

XXI. The drape assembly of clause XX, wherein the first guide interface and the second guide interface are each shaped to releasable engage one of the first keeper interface and the second keeper interface.

XXII. A robotic surgical system comprising:
a robotic arm having a plurality of links between a distal end and a proximal end;
an end effector operatively attached to the distal end of the robotic arm, the end effector including a plurality of markers protruding from a surface of the end effector; and
a drape including:
   a first portion defining an opening shaped to receive the distal end of the robotic arm therethrough, and
   a second portion disposed in fluid communication with the first portion and defining a cavity shaped to accommodate at least a portion of the distal end of the robotic arm therein, the second portion of the drape being formed from an elastic polyisoprene material shaped to cover the end effector and contact each of the plurality of markers.

XXIII. The robotic surgical system of clause XXII, further comprising a navigation system configured to optically track the plurality of markers on the end effector through the second portion of the drape.

XXIV. The robotic surgical system of any of clauses XXII-XXIII, wherein each of the plurality of markers includes a lens, with the second portion of the drape contacting and covering the lens of each of the plurality of markers.

XXV. The robotic surgical system of any of clauses XXII-XXIV, wherein each of the plurality of markers includes a light emitting diode.

XXVI. The robotic surgical system of any of clauses XXII-XXV, further comprising a keeper coupled to the robotic arm, the keeper having a keeper interface; and
wherein the drape further includes a guide disposed between the first portion and the second portion and having a guide interface configured to releasably attach to the keeper interface of the keeper to secure the drape to the robotic arm.

XXVII. The robotic surgical system of clause XXVI, wherein the guide defines an aperture having an inner perimeter larger than an outer profile of the keeper.

XXVIII. The robotic surgical system of clause XXVII, wherein the guide defines an airflow channel arranged between the outer profile of the keeper and the inner perimeter of the aperture of the guide.

XXIX. A method of using a robotic surgical system, the method comprising;
positioning an end effector of a robotic arm within a field of view of a localizer of a navigation system, the end effector having a plurality of markers;
covering the end effector with a drape formed from an elastic polyisoprene material in contact with each of the plurality of markers; and
detecting, with the localizer of the navigation system, light emitted by the plurality of markers through the drape.

1. A robotic surgical system comprising:
a robotic arm extending between a distal end and a proximal end;
a keeper coupled to the robotic arm, the keeper having a keeper interface; and
a drape including:
a first portion defining an opening shaped to receive the distal end of the robotic arm therethrough,
a second portion disposed in fluid communication with the first portion and defining a cavity shaped to accommodate at least a portion of the distal end of the robotic arm therein, and
a guide disposed between the first portion and the second portion and having a guide interface configured to releasably attach to the keeper interface of the keeper to secure the drape to the robotic arm.

2. The robotic surgical system of claim 1, wherein the keeper interface of the keeper includes a seat; and
wherein the guide includes a retainer operatively attached to the guide and shaped to releasably engage the seat of the keeper.

3. The robotic surgical system of claim 1, wherein the guide is at least partially formed from a resilient material.

4. The robotic surgical system of claim 3, wherein the keeper interface includes a taper having a ramped profile shaped to resiliently enlarge the guide as the guide is passed over the keeper.

5. The robotic surgical system of claim 1, wherein the guide defines an aperture having an inner perimeter larger than an outer profile of the keeper.

6. The robotic surgical system of claim 5, wherein the guide defines an airflow channel arranged between the outer profile of the keeper and the inner perimeter of the aperture of the guide.

7. The robotic surgical system of claim 1, further comprising an end effector operatively attached to the distal end of the robotic arm, the end effector including a plurality of markers protruding from a surface of the end effector; and
wherein the second portion of the drape is formed from an elastic polyisoprene material shaped to cover the end effector and contact each of the plurality of markers.

8. The robotic surgical system of claim 7, further comprising a navigation system configured to optically track the plurality of markers on the end effector through the second portion of the drape.

9. The robotic surgical system of claim 1, wherein the first portion of the drape and the second portion of the drape are formed from different materials; and
wherein the second portion of the drape is formed from an elastic polyisoprene material.

10. (canceled)

11. The robotic surgical system of claim 1, wherein the first portion of the drape and the second portion of the drape are formed from the same material.

12. The robotic surgical system of claim 1, wherein the first portion of the drape includes:
a distal section coupled to the guide, and
a proximal section defining the opening.

13. The robotic surgical system of claim 1, wherein the keeper includes two keeper interfaces; and
wherein the guide includes two guide interfaces to respectively releasably attach to the two keeper interfaces.

14. The robotic surgical system of claim 1, wherein the keeper is integrated into the robotic arm adjacent to the distal end.

15. The robotic surgical system of claim 1, wherein the keeper is removably connected to the robotic arm adjacent to the distal end.

16. (canceled)

17. (canceled)

18. (canceled)

19. (canceled)

20. (canceled)

21. A robotic surgical system comprising:
a robotic arm having a plurality of links between a distal end and a proximal end;
an end effector operatively attached to the distal end of the robotic arm, the end effector including a plurality of markers protruding from a surface of the end effector; and
a drape including:
a first portion defining an opening shaped to receive the distal end of the robotic arm therethrough, and
a second portion disposed in fluid communication with the first portion and defining a cavity shaped to accommodate at least a portion of the distal end of the robotic arm therein, the second portion of the drape being formed from an elastic polyisoprene material shaped to cover the end effector and contact each of the plurality of markers.

22. The robotic surgical system of claim 21, further comprising a navigation system configured to optically track the plurality of markers on the end effector through the second portion of the drape.

23. The robotic surgical system of claim 21, wherein each of the plurality of markers includes a lens, with the second portion of the drape contacting and covering the lens of each of the plurality of markers.

24. The robotic surgical system of claim 21, wherein each of the plurality of markers includes a light emitting diode.

25. The robotic surgical system of claim 21, further comprising a keeper coupled to the robotic arm, the keeper having a keeper interface; and
wherein the drape further includes a guide disposed between the first portion and the second portion and having a guide interface configured to releasably attach to the keeper interface of the keeper to secure the drape to the robotic arm.

26. The robotic surgical system of claim 25, wherein the guide defines an aperture having an inner perimeter larger than an outer profile of the keeper; and
wherein the guide defines an airflow channel arranged between the outer profile of the keeper and the inner perimeter of the aperture of the guide.

27. (canceled)

28. (canceled)
